(19) United States
(12) Patent Application Publication
(10) Pub. No.: US 2016/0083773 A1
(43) Pub. Date: Mar. 24, 2016

(54) Apparatus and Process for Treating Samples of Biological or Microbiological Material

(71) Applicant: COPAN INFORMATION TECHNOLOGIES S.R.L., Brescia (IT)

(72) Inventor: Daniele Triva, Bovezzo (Brescia) (IT)

(21) Appl. No.: 14/891,607

(22) PCT Filed: Apr. 30, 2014
(86) PCT No.: PCT/IB2014/061109

§ 371 Date: Nov. 16, 2015

(30) Foreign Application Priority Data
May 17, 2013 (IT) MI2013A000812

(51) Int. Cl. C12Q 1/24

(52) U.S. Cl. CPC C12Q 1/24

(57) ABSTRACT

An apparatus for treating samples includes a first housing interface (3) for a culture support (2), a biological or microbiological material (16) being arranged on the culture support (2); a first computer (9) with a processor and a memory unit; a visual signaling device (11), operatively connected to the first computer (9); a first software program (14) stored and operating on the first computer (9) and configured such as to process a memorized first image (15) of the culture support (2) and for determining a collecting position (12) of a biological or microbiological material (16) in the first image (15), the first software program (14) being configured to control functioning of the visual signaling device (11) such as to emit a light signal on the culture support (2) housed in the first interface (3) such as to visually indicate the collecting position (12) of a biological or microbiological material (16) on the culture support (2), for guiding a collection of a sample of the biological or microbiological material (16) at the collecting position (12) by an operator.

The present invention relates to an apparatus and a process for treating samples of biological or microbiological material. The invention is particularly applicable in the health, clinical and environmental sectors, as well as in all cases where samples of biological, microbiological, bacteriological types are to be subjected to successive analysis.

The prior art includes use of various types of supports of cellular and/or bacteriological culture, such as for example Petri dishes or capsules, constituted by a flat glass recipient or more often a flat plastic recipient, usually transparent and cylindrical in shape. A Petri dish is substantially a container for solid and semi-solid culture media, and can be used in various laboratory operations, in particular for cultivating cellular colonies, in particular bacterial, using various techniques. Petri dishes provided with culture media are generally seeded with biological or microbiological samples, after which they are subjected to an incubation period, enabling growth of cellular colonies, for example bacterial. The bacterial colonies can first be observed for their morphological characteristics, and are generally subjected to further specific analyses with the aim of precisely identifying the type of cells and/or bacteria, present in the initial sample.

It is known to collect cellular samples of isolated colonies present in the Petri dish so as to subject the colonies to further specific analyses. The sample collection is in general carried out by a laboratory operator at a position previously indicated by a medical doctor or a highly-specialised operator in the analysis of cellular and/or bacterial growths. In the prior art, this identification is carried out very approximately, for example by application of an identifying sign below the Petri dish realized with a marker pen or a like tool.

This operating method exhibits very significant limitations, as it is characterised by poor repeatability and a considerable imprecision in the identification of the collection point desired by the medical doctor, with considerable risks of error in the correct identification of the collection point and therefore in the collecting of the sample desired by the doctor. The known method is therefore at great risk of analytical errors, with consequently potentially serious consequences for patients' health. The samples collected from the Petri dishes can then be analysed using various types of analysis, for example also by positioning of the samples on matrix transport supports provided with a plurality of depositing sites, in which the samples can be seeded or arranged using instruments which automatically perform the examination of the sample deposited on the matrix supports. Examples of these transport supports are constituted by the plates used for mass spectrometry examinations, such as MALDI (Matrix-Assisted Laser Desorption/Ionization) or MALDI-TOF (Time of Flight), by anti-biogram support, for food tests, environmental analysis, by plates and micro-plates for molecular biology, etc. Various types of such transport devices are known, having a support surface provided by a plurality of sites on which the samples of materials to be analysed are seeded or arranged using conventional techniques. The transport support sites can be fashioned on the surface of the support (for example by incision), can simply be visually delimited by an edge delimiting each site, or may not be defined significantly before the use of the transport supports, and therefore be simply constituted by positions on the support surface (identifiable only by specific coordinates in terms of y and x axes) which are in no way delimited or predefined before the depositing of sample and are only defined by the seeding and depositing of the sample.

The samples deposited on the transport support are then subjected to specific examination procedures or analyses which enable determination of some specific properties of the samples. Each sample, in relation to a determined type of material having a determined origin (for example a specific patient or a collecting site), is seeded or deposited on the transport support in a precise position or site, then to be subjected to laboratory examination of an analytic or diagnostic type.

In all of the above-cited cases, it is of fundamental importance that a record is kept of the precise positioning of each sample on the transport support, such as to be able to correctly pair the analysis results of each sample with the correct origin thereof. An error in positioning of the samples of the transport support and/or in the previous or successive pairing of the analysis results with the patients or the sources of the samples can have serious consequences, as it might lead to swapping results of the analyses and therefore to very serious errors, producing false positives or false negatives. In order to reduce these risks, it is known to predispose control protocols which are in general prepared before depositing the samples on the transport supports or targets, in which various data relating to each sample are entered, for example the nature and origin of the sample, and in which an order or design of distribution of the samples on the target or transport support is included. The operators depositing the samples on the target must therefore relate to the control protocols and follow them scrupulously so as to prevent error. Also known are computer-based systems and software programs that aid the operators in the preparation of the control protocols, which are however always manually compiled by the operators, and enable printing out the control protocols for use by the operators when depositing the samples.

Alternatively the operators can simply deposit the samples in a progressive order and consequently compile the control protocols, reporting therein the tasks performed. In any case, after the carrying-out of the analysis of the samples, the results thereof are associated to the various patients or the sources of the samples in accordance with the established order in the control protocols. The Applicant has found that the known solutions described in the foregoing are strongly exposed to the risk of human error both during the compilation of the control protocols and during the procedure of depositing the samples on the support devices, as well as during the following step of matching the results of the analyses to the sources of the samples. In fact, notwithstanding the experience of the operators themselves, the use of management software programs and the editing of the control protocols, it is constantly possible for an operator to make an error in one of the cited steps, and therefore the above-described solutions are intrinsically not secure and potentially subject to grave risks for the patients and for the individuals involved in the analysis. Further, the above-described solutions are often complex, laborious and require very prepared and careful personnel and do not enable a sufficient traceability of the whole process carried out and the case history relative to the analyses carried out, the devices used and the subjects involved, thus losing a source of potentially very important data in some specific contexts.

A main aim of the present invention is to obviate one or more of the drawbacks encountered in the prior art.

An aim of the present invention is to provide an apparatus and a process for treatment of samples of biological or microbiological material which enable eliminating or at least significantly reducing the risk of human error in identifying the samples to be analysed, in carrying out the analysis processes and/or in determining the results thereof.

A further aim of the present invention is to provide an apparatus and a process for treating samples exhibiting a high degree of reliability and repeatability of the results.

A further aim of the present invention is to provide an apparatus and a process for treating samples which are very flexible and adaptable to various operations needs and different types of analysis.

A further aim of the present invention is to provide an apparatus and a process for treatment of samples or analytes which enable a simplification and acceleration of the treatment processes of the data relative to the analyses of the samples.

A further aim of the present invention is to provide an apparatus and a process for treatment of samples which offer a high degree of traceability of the historical data relative to the analyses carried out and the devices and subjects involved, so as to enable further study and statistics relating to the results.

A further aim of the present invention is to disclose an apparatus and a process for treating samples which enables significantly increasing analysis procedures of samples or analyses, the safety of patients, and in general the safety of the various subjects involved in the analyses.

A further aim of the present invention is to disclose an apparatus and a process for treatment of samples or analytes which are simple to realize and not expensive to make.

The aims and others besides, which will more fully emerge from the following description, are substantially attained by an apparatus and a process for treatment of samples of biological or microbiological material, according to what is set out in one or more of the appended claims, taken alone or in combination, or in any combination with one or more of the further aspects described in the following.

Each of the aspects described in the following can further be taken alone or in any combination with the other described aspects, and further also in combination with any one of the claims of the present application or any combination of the claims.

In an aspect, the invention relates to an apparatus for treatment of samples of biological or microbiological material, wherein the apparatus comprises at least: a first housing interface for a culture support, in particular a Petri dish, at least a biological or microbiological material being arranged on the culture support; a first computer provided at least with a processor and a memory unit; at least a visual signaling device, operatively connected to the first computer; at least a first software program stored and operating on the first computer and configured such as to process at least a memorized first image of the culture support and for determining at least a collecting position of a biological or microbiological material in the first image, the first software program being configured and operatively active further to control functioning of the visual signaling device such as to emit at least a light signal on the culture support housed in the first interface such as to visually indicate at least the collecting position of a biological or microbiological material on the culture support, for guiding a collection of a sample of the biological or microbiological material at the collecting position by an operator.

The cellular and/or bacterial support, such as for example a Petri dish or a Petri capsule, is constituted by a flat glass or plastic recipient that is usually transparent and usually cylindrical. The culture support is substantially a container for solid and semi-solid culture media, comprising for example agar, and can be used in various laboratory operations, in particular for cultivating cellular colonies, in particular bacterial, with various methods. The culture supports most used have a diameter comprised between about 35 mm and about 100 mm and a height of about 10-15 mm. The culture surface of a culture support is comprised between about 5 cm² and about 80 cm². The sample of the biological or microbiological material collected is a very limited portion of the above surface, usually comprised between 0.1 mm² and 10 mm².

In an aspect, the apparatus is configured such as to receive the first image with the collecting position already previously selected by a user by means of a second software program operating on a second computer.

In an aspect the first software program is configured so as to display and enable a user to select the collecting position on the first memorized image on the culture support.

In an aspect, the first software program, or the second software program, is configured so as automatically to define, for enabling manually defining, dimensions and/or a profile of a predetermined shape about the collecting position.

In an aspect, the invention further relates to an apparatus for biological or microbiological material in which a first interface and a second interface are selectively mountable to a same base body of the apparatus.

In a further aspect, the invention further relates to an apparatus in which the first interface comprises at least a first housing site complementarily-shaped to a culture support, in particular a Petri dish and/or wherein the second interface comprises at least a second housing site complementarily-shaped to a transport support, in particular a plate of the MALDI type.

In a further aspect, the invention further relates to an apparatus in which the first interface can be replaced with at least a third interface suited to a different type of culture support and/or in which the second interface is replaceable with at least a fourth interface suited to a different type of transport support.

In a further aspect, the invention further relates to an apparatus in which the first interface and the second interface are realized and defined in a same base body.

In a further aspect, the invention further relates to an apparatus in which the visual signaling device is mounted on a support body of the apparatus.

In an aspect, the visual signaling device is a laser pointer device.

In an aspect, the visual signaling device is configured and predisposed to emit a laser beam selectively indicating the collecting position on the culture support.

In an aspect, the visual signaling device is aimed at generating a laser beam pointed at least towards a proximal position to and distinct from the collecting position, preferably towards a plurality of positions proximal to and distinct from the collecting position.

In an aspect, the visual signaling device is aimed at generating a laser beam defining a predetermined shape, preferably circular, developing about the collecting position.

In an aspect, the visual signaling device is automated and/motorized so as to selectively vary the collecting position indicated on the culture support.

In an aspect, the predetermined shape is defined statically by the laser beam.

In an aspect, the predetermined shape is defined dynamically by the laser beam.

In a further aspect, the invention relates to an apparatus in which the visual signaling device is a laser pointer mounted rotatably on a support body so as to be able to rotate about at least a rotation axis, and preferably about at least two perpendicular rotation axes or by means of a ball joint, so as to vary the collecting position indicated on the culture support.

In an aspect, the first housing interface is provided at least with a reference indicator aimed at enabling a precise positioning according to a defined orientation of the culture support on the first housing interface.

In an aspect, the reference indicator is determined such as to enable arranging a reference element, preferably a bar code, of the culture support at the reference element with the culture support arranged in the first housing interface.

In an aspect, the apparatus comprises at least a first camera arranged at least at the first interface and connected to the first computer so as to provide the first software program with a second reference image of the culture support housed in the first interface, the first software program being configured so as to compare the first image and the second image and to automatically determine, or enable an operator to manually determine, a corrective factor preferably of angular positioning of the culture support in the first image with respect to the second image.

In an aspect, the first software program is configured such as to automatically correct the collecting position indicated by the signaling device on the culture support in consideration of the corrective factor. In an aspect, the apparatus further comprises an automatic movement device of the culture support housed in the first interface such as to correct the angular position thereof so that the positioning in a third image detected by the first camera corresponds to the positioning thereof in the first image.

In an aspect, the apparatus comprises at least a display connected to the computer and commanded by the first software program for displaying at least the first image and/or the second image and/or the third image of the culture support.

In an aspect, the apparatus further comprises at least a second housing interface for a transport support for samples of biological or microbiological material.

In an aspect, the transport support is provided with a plurality of depositing positions or sites able each to receive at least a sample of biological or microbiological material.

In an aspect, in which the visual signaling device is commanded by the first software program of the first computer so as further to indicate at least a depositing position or site of the samples on the transport support or for progressively indicating the depositing positions or sites of the samples on the transport support.

In an aspect, the visual signaling device is configured and predisposed to emit a laser beam selectively indicating a depositing position or site or a plurality of depositing positions or sites on the transport support.

In a further aspect, the invention further relates to an apparatus in which the base body is configured such as to be able to house a plurality of interfaces each adapted to a specific different type of culture support.

In a further aspect, the invention further relates to an apparatus in which the base body and the support body are solidly constrained to one another.

In a further aspect, the invention further relates to an apparatus further comprising a culture support, for example a Petri dish, provided with a culture media for samples of biological or microbiological material and at least a biological or microbiological material in the medium, the culture support being housable on the first housing interface.

In a further aspect, the invention further relates to an apparatus in which the software program is predisposed and configured such as to singly identify each culture support by means of manual insertion of an identifying datum only of the specific culture support or by means of reading, using of a suitable bar code reader, a bar code applied to the culture support.

In a further aspect, the invention further relates to an apparatus further comprising a transport support for samples of biological or microbiological material, the transport support being selectively housable in the second housing interface.

In a further aspect, the invention further relates to an apparatus in which the visual signaling device is aimed at emitting the light signal onto the transport support for selectively indicating the effect depositing position or site.

In a further aspect, the invention further relates to an apparatus in which the visual signaling device is controlled by the first software program so as to indicate a single positioning site at a time.

In a further aspect, the invention further relates to an apparatus in which the visual signaling device is controlled by the first software program so as to simultaneously indicate at least two of the above-mentioned positioning sites.

In a further aspect, the invention further relates to an apparatus in which the transport support further comprises an electronic storage device, recordable and is aimed at memorising a plurality of data relating at least to the material samples and the specific arrangement of each of the material samples on the transport support, at each of the depositing sites, the electronic storage device being constrained to the transport support.

In a further aspect, the invention further relates to an apparatus in which the storage device is cancellable and rewritable a plurality of times so as to enable selectively cancelling and rewriting the plurality of data and/or wherein the storage device is further provided with at least a unique and individual identifying datum relating to a technical characteristic of the transport support, selective from at least the following: the type of support, the type of support surface, the number of depositing sites of the support, the arrangement of depositing sites on the support, the coordinates of the depositing sites on the support, the presence or not of culture media on the support and/or wherein the storage device comprises at least a portion of non-rewritable memory in which the cited at least a single and individual identifying datum of the transport support and/or the at least a datum relative to a technical characteristic of the transport support is memorized.

In a further aspect, the invention further relates to an apparatus in which the storage device comprises at least a TAG or a recordable RFID transponder, provided at least with an electronic memory chip and an RFID antenna and/or wherein the storage device is a TAG or an RFID transponder of a passive type and/or wherein storage device is integrated in the transport support and/or wherein the transport support exhibits a flat, continuous and non-porous surface at and in proximity of the mounting point of the storage device on the transport support.

In a further aspect, the invention further relates to an apparatus in which the second interface further comprises at least a transmission device aimed at reading and modifying the contents of the electronic storage device of the transport support and/or comprises at least an RFID transceiver provided with at least an antenna for reading and/or writing the contents of the electronic storage device, of the RFID type.

In a further aspect, the invention further relates to an apparatus further comprising at least a first camera for recording images at least of the transport support during the positioning of a plurality of samples of material and/or wherein the first camera is connected to the first computer for transferring the images to the first computer, which is aimed at conserving and/or verifying the images to confirm the real arrangement of the plurality of samples of material on the transport support.

In a further aspect, the invention further relates to an apparatus in which a first software program is aimed at using a single and individual datum of the transport support and/or one or more items of data relating to technical characteristics of the transport support at least so as to further show on a display a representation of the transport support and the relative arranged on the depositing sites.

In a further aspect, the invention further relates to an apparatus in which the first software program is aimed at using a single and individual datum of the transport support and/or one or more items of data relating to technical characteristics of the transport support at least so as to actuate an automatic configuration procedure and/or a guide procedure of the visual signaling device.

In a further aspect, the invention further relates to an apparatus in which the apparatus is predisposed and configured so as to obtain the single and individual datum of the transport support by means of manual insertion of the datum in the first computer or by means of a reading of a bar code applied to the transport device.

In a further aspect, the invention further relates to an apparatus in which an electronic storage device of the illustrated type is applied to the culture support in a like way to what is indicated for the transport support.

In a further aspect, the invention further relates to use of an apparatus, according to any one of the claims of the above-indicated aspects, for treatment of samples of biological or microbiological material in a diagnostic, medical, analytical, chemical, environmental, food and/or industrial field.

In an aspect, the invention relates to a process for treating samples of biological or microbiological material, comprising at least steps of: predisposing a culture support, in particular a Petri dish, exhibiting biological or microbiological material grown there; obtaining at least a first image of the culture support; selecting, in the first image, a collecting position of a sample of the biological or microbiological material; arranging the culture support in a first housing interface of an apparatus for treatment of samples of biological or microbiological material; projecting on the culture support housed in the first interface at least a light signal able to visually indicate the collecting position of the biological or microbiological material on the culture support; and collecting the sample of the biological or microbiological material from the culture support at the collecting position.

In an aspect, the steps of obtaining the first image and selecting in the first image the collecting position are carried out by means of a preliminary computer and a preliminary software program by a highly-specialized operator.

In an aspect, the first image with the collecting position selected is transferred from the apparatus before projecting the light signal onto the culture support.

In an aspect, the process comprises one or more of following steps:

automatically, or manually, defining dimensions and/or an outline of a predetermined shape to be projected by means of the light signal about the collecting position arranged in the first interface.

In a further aspect, the invention further relates to a process for treating samples of biological or microbiological material in which the light signal is a laser beam.

In a further aspect, the invention further relates to a process in which the laser beam is pointed towards at least a position close to and distinct from the collecting position, preferably towards a plurality of positions proximal to and distinct from the collecting position.

In a further aspect, the invention further relates to a process in which the visual signaling device is aimed at generating a laser beam defining a predetermined shape, for example circular, developing about the collecting position.

In a further aspect, the invention further relates to a process in which the predetermined shape is defined statically by the laser beam.

In a further aspect, the invention further relates to a process in which the predetermined shape is defined dynamically by the laser beam and/or i.e. by means of a mobile laser beam about the collecting position.

In an aspect, the process comprises: obtaining at least the second reference image of the culture support housed in the first interface and comparing the first image with the second image; automatically or manually determining a corrective factor of angular positioning of the culture support in the first image with respect to the second image; automatically correcting the collecting position indicated by the signaling device on the culture support in consideration of the corrective factor; automatically or manually correcting the angular position of the culture support in the first interface such as to annul the corrective factor and make the angular position of the culture support coincide in a third image detected by the first camera with the corresponding angular position of the culture support in the first image.

In a further aspect thereof, the invention further relates to a process further comprising a step of visualizing at least the first image and/or the second image and/or the third image of the culture support on a display.

In a further aspect, further relaters to a process further comprising a step of arranging a reference element, preferably a bar code, of the culture support, at the reference element with the culture support arranged in the first housing interface, so as to precisely determine the angular position of the culture support on the first interface.

In a further aspect, the invention further relates to a process further comprising the step of identifying the culture support by means of reading a bar code or by manually inserting a single datum relating to the culture support taken for example from an identifying label of the culture support.

In a further aspect thereof, the invention further relates to a process further comprising a step of arranging a plurality of samples of biological or microbiological material on a corresponding plurality of depositing sites of a transport device housed in a second interface and projecting on the transport support at least a light signal, during the step of arranging the plurality of samples of material, at the effective depositing position or depositing site or depositing sites in which each sample of material is arranged, so as to guide the position or for visibly indicating the positioning.

In a further aspect thereof, the invention further relates to a process further comprising the step of projecting the light signal on the transport signal selectively at the effective depositing position or site or depositing sites and/or further comprising a step of progressively indicating the depositing positions or sites of the samples on the transport support, by means of the light signal, indicating a single positioning site at a time or simultaneously indicating at least two of the positioning sites.

In a further aspect thereof, the invention further relates to a process further comprising a step of storing on an electronic storage device, constrained to the transport support, a plurality of data relative to at least the samples of biological or microbiological samples and the specific arrangement of each thereof on the transport support, at the depositing sites; and/or reading from the electronic storage device, constrained to the transport support so as to be transportable together with the transport support, at least a single and individual identifying datum of the transport support and/or at least a datum relating to a technical characteristic of the transport support and/or a plurality of data relating at least to the samples of biological or microbiological material and to the specific arrangement to be carried out for each thereof on the transport support, at the depositing sites.

In a further aspect thereof, the invention further relates to a process further comprising one or more of the following steps: register images or photographs at least of a part of the transport support during the step of arranging a plurality of samples of material, and conserving and/or verifying the images so as to keep a trace of the real arrangement of the plurality of samples of material on the transport support; using a single and individual identifying datum of the transport support, and/or of the culture support and/or one or more items of data relating to technical characteristics of the transport support and/or of the culture support, in the first software program; using a single and individual identifying datum of the transport support and/or one or more items of data relating to technical characteristics of the transport support in a software program aimed at visualizing on a display a representation of the transport support and the relative arrangement of the depositing sites or in a process of automatic configuration and guide configuration of the visual signally device; memorizing on an electronic storage device, constrained to the transport support, a plurality of data relative at least to the samples of biological or microbiological material and the specific arrangement of each thereof on the transport support, at the depositing sites and/or wherein the electronic storage device comprises at least a TAG or a recordable RFID transponder, provided at least with an electronic memory chip and an RFID antenna and/or wherein the storage device is a TAG or an RFID transponder of a passive type; reading from the electronic storage device, constrained to the transport device, at least a single and individual identifying datum of the transport support and/or at least a datum relating to a technical characteristic of the transport support and/or a plurality of data relating at least to the samples of biological or microbiological material and the specific arrangement to be carried out for each thereof on the transport sites; memorizing on the storage device and/or in the first computer the results of the analyses carried out on the samples deposited on the transport support, in combination corresponding to the plurality of information on the samples and on the arrangement thereof on the transport support.

A detailed description of one or more preferred embodiments of the invention will now be given, by way of non-limiting example, in which:

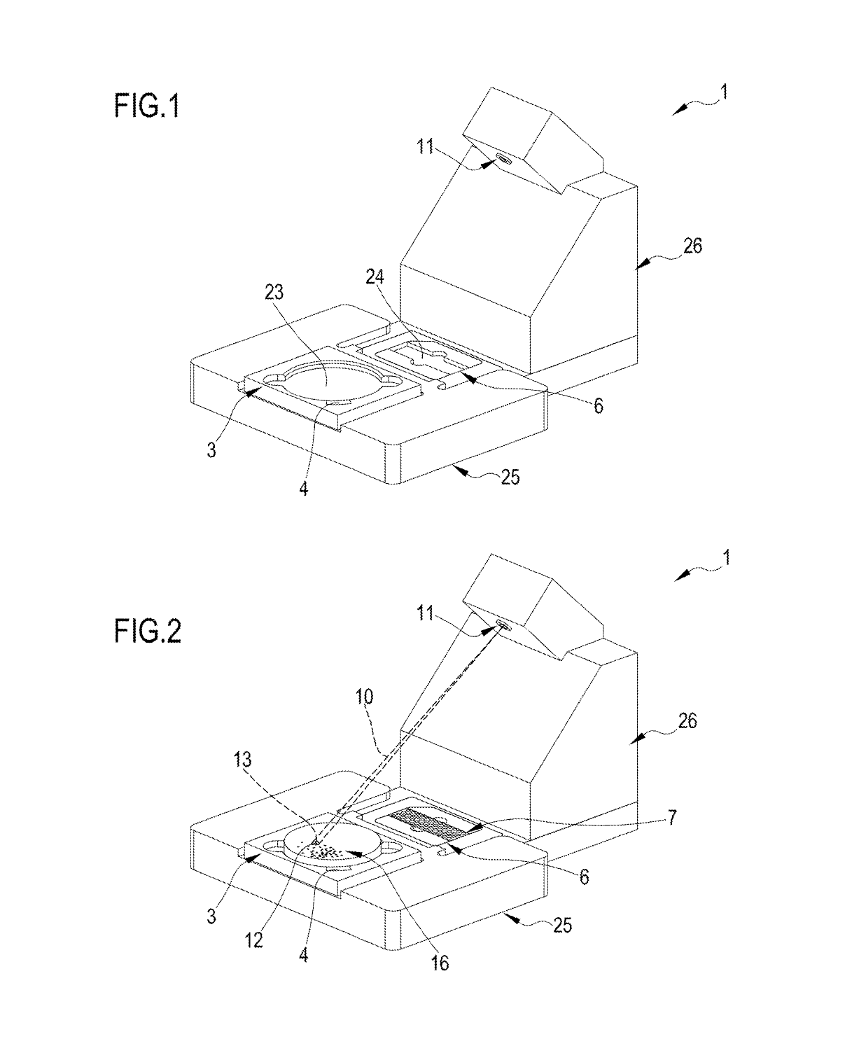

FIG. 1 is a perspective view from above of an apparatus for treatment of samples of biological and/or microbiological material according to a first embodiment;

FIG. 2 shows the apparatus of FIG. 1 with a culture support and a transport support housed in relative housing interfaces, with a laser beam indicating a collecting position on the culture support;

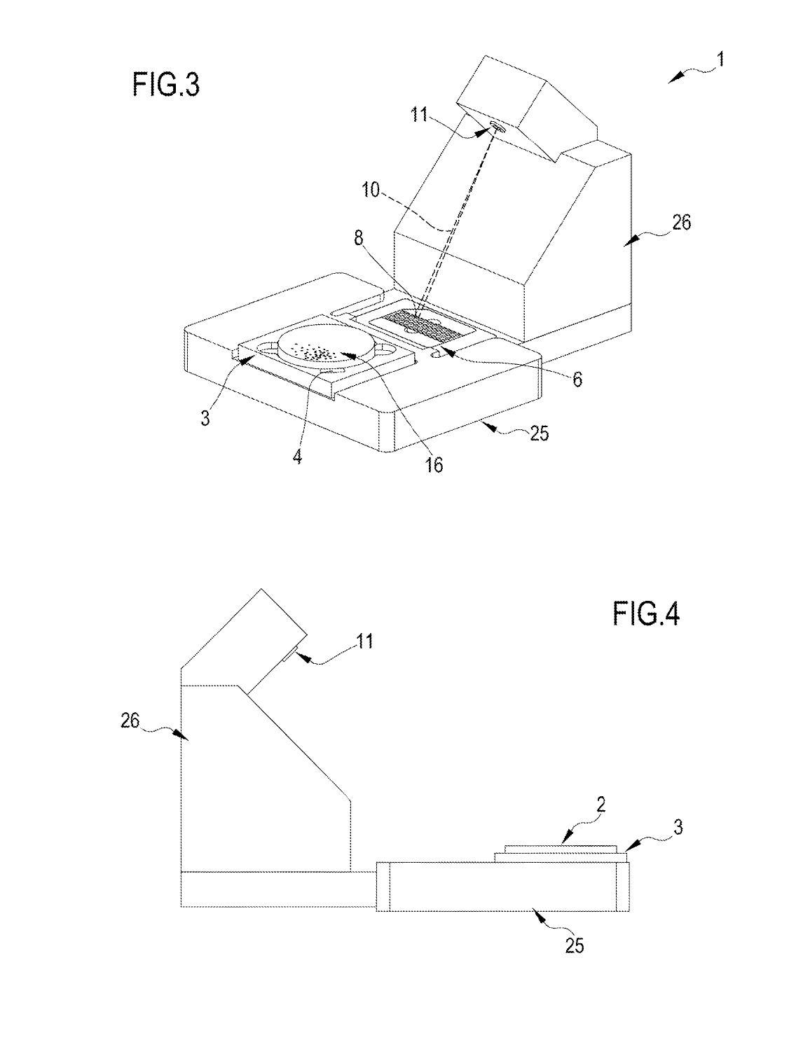

FIG. 3 is a view as in FIG. 1, in which the laser beam indicates a depositing site or position of a sample on the transport support;

FIG. 4 is a lateral view of the apparatus of FIG. 1;

FIG. 5 is a view from above of the apparatus of FIG. 1;

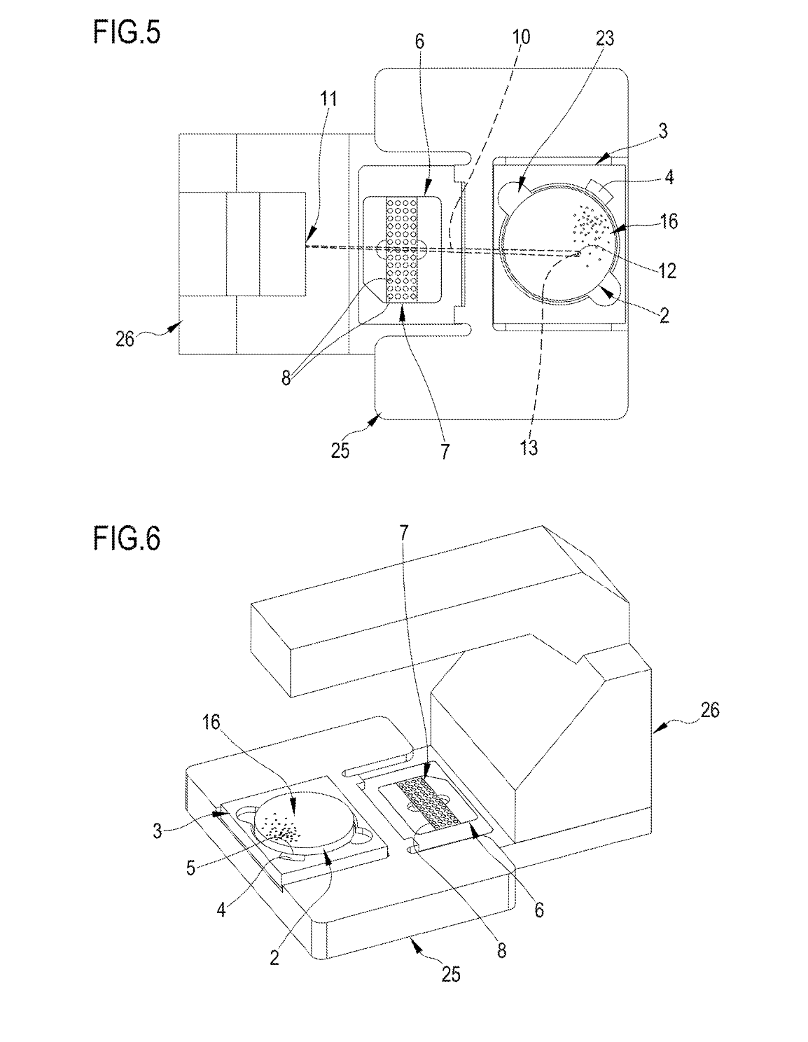

FIG. 6 is a second embodiment of an apparatus for treating samples of biological and/or microbiological material;

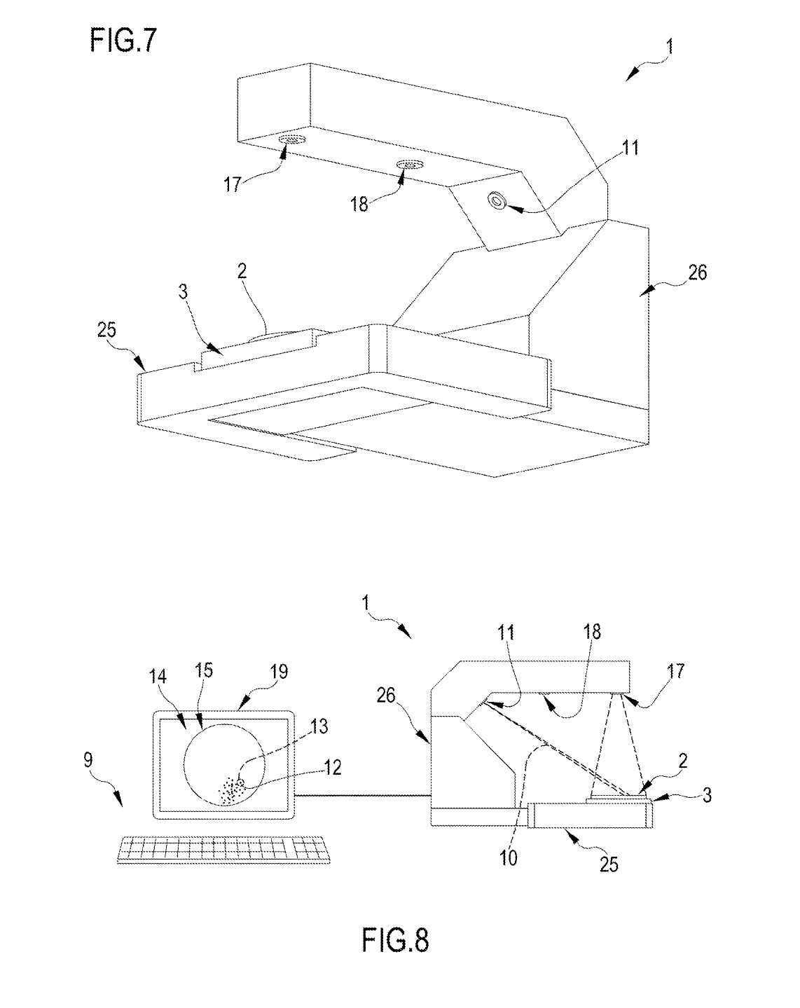

FIG. 7 is a perspective view from below of the apparatus of FIG. 6;

FIG. 8 is a lateral view of the apparatus of FIG. 6, with further components such as a computer and a display;

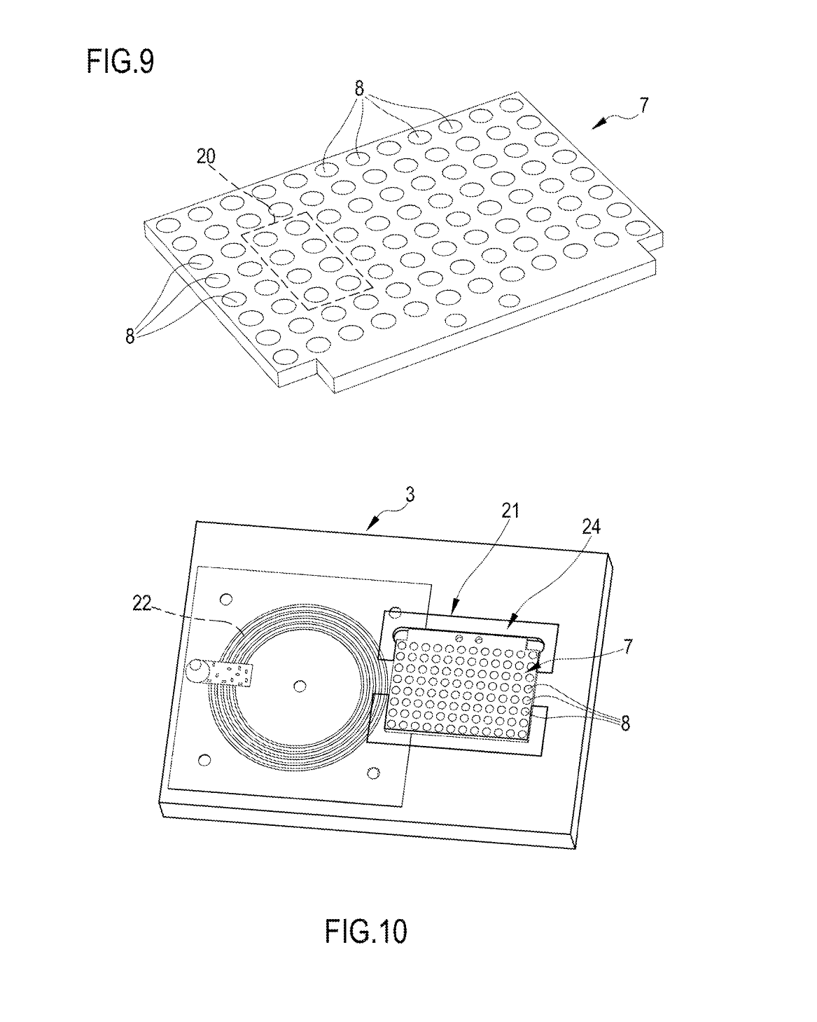

FIG. 9 is a detail of an embodiment of a transport support;

FIG. 10 is a detail of the transport support of FIG. 9, housed in a second interface according to an alternative embodiment.

An apparatus 1 will be described for treating samples of biological and/or microbiological material 16, according to some embodiments of the invention. The apparatus 1 comprises at least a first interface 3 for housing a culture support 2, in particular a Petri dish, at least a biological or microbiological material being arranged on the culture support 2. The term “biological” material is intended to extend also to include material of a biological origin, and by “biological and/or microbiological material” are intended both biological material comprising microbiological material and purely microbiological material, such as for example bacteria. The first housing interface 3 is preferably provided at least with a reference indicator 4 aimed at enabling a precise positioning according to a defined orientation of the culture support 2 on the first housing interface 3. The reference indicator 4 can be determined such as to enable arranging a reference element 5, preferably a bar code, of the culture support 2, at the reference element 5 with the culture support 2 arranged in the first housing interface 3. In other terms, the bar code can be aligned with the reference indicator 4 so as to define the correct angular position of the culture support 2 on the first interface 3.

The apparatus 1 can further comprise at least a second housing interface 6 for a transport support 7 for samples of biological or microbiological material 16. The transport support 7 is provided with a plurality of depositing positions or sites 8 able each to receive at least a sample of biological or microbiological material 16. The first interface 3 can comprise at least a first housing sites 23 complementarily shaped with a culture support 2, in particular a Petri dish. The second interface 6 can comprise at least a second housing site 24 complementarily-shaped to a transport support 7, in particular a MALDI plate. The first interface 3 can be replaceable with at least a third interface suitable for a different type of culture support 2. The second interface 6 can be replaceable with at least a fourth interface suitable for a different type of transport support 7. The first interface 3 and the second interface 6 can be realized and defined in a same base body 25.

The base body can be configured so as to be able to house a plurality of interfaces each suited to a specific different type of culture support 2.

The apparatus 1 further comprises at least a first computer 9 provided at least with a processor and a memory unit.

The apparatus 1 further comprises at least a visual signaling device 11, operatively connected to the first computer 9. The visual signaling device 11 can be mounted on a support body 26 of the apparatus 1. The base body and the support body can be solidly constrained or separate. The visual signaling device 11 can be a laser pointer device and/or can be configured and predisposed to emit a light signal 10, for example and preferably a laser beam 10, selectively indicating the collecting position 12 on the culture support 2. The visual signaling device 11 is preferably automated and/or motorized so as to selectively vary the collecting position 12 indicated on the culture support 2. The visual signaling device 11 is preferably aimed at generating a laser beam 10 pointed towards at least a position proximal to and distinct from the collecting position 12, preferably towards a plurality of positions proximal and distinct from the collecting position 12.

The visual signaling device 11 can be a laser pointer mounted rotatably on a support body so as to be rotatable about at least a rotation axis, and preferably about at least two perpendicular rotation axes or by means of a ball joint, so as to vary the collecting position 12 indicated on the culture support 2.

The visual signaling device 11 is preferably aimed at generating a laser beam 10 defining a predetermined shape, preferably circular, developing about the collecting position 12 (as visible for example in FIGS. 2 and 5). The predetermined shape 13 can be defined statically by the laser beam 10, i.e. with a fixed laser beam 10, or can be defined dynamically by the laser beam 10. i.e. the laser beam can move so as to describe the shape 13 dynamically. In this way it is possible to avoid pointing the laser beam 10 directly on the collecting point, providing important advantages, such as to avoid risking damaging the sample to be collected and enabling the operator performing the collecting to see, precisely and without visual disturbance, the appearance of the sample in the collecting point.

The visual signaling device 11 can be commanded by a first software program 14 of the first computer 9 such as further to indicate at least a depositing position or site of the samples on the transport support 7 housed on the second interface 6 or to progressively indicate the depositing positions or sites 8 of the samples on the transport support 7, so as to guide the positioning of the sample of material or so as to define and delimit the plurality of depositing sites 8 on the transport support 7 (substantially describing a light network defining the sites) and/or so as to visibly indicate the positioning. The visual signaling device 11 can further be configured or predisposed to as to emit the laser beam 10 selectively indicating a depositing position or site or a plurality of positions of depositing positions or sites 8 on the transport support 7.

The apparatus 1 further comprises at least a first software program 14 memorized and operating on the first computer 9 and configured so as to process at least first memorized image 15 of the culture support 2 and so as to determine at least a collecting position 12 of a biological or microbiological material 16 in the first image 15.

The first software program 14 can be configured and operatively active further to control the functioning of the visual signaling device 11 so as to emit at least a light signal on the culture support 2 housed in the first interface 3 so as to visually indicate at least the collecting position 12 of a biological or microbiological material 16 on the culture support 2, so as to guide a collecting of a sample of the biological or microbiological material 16, by an operator, at the collecting position 12.

The first software program 14, or a second software program operating on the first computer 9 or on a second computer, can be configured such as to display and enable a user to select the collecting position 12 on the first visual image 15 memorized on the culture support 2, such that the visual signaling device 11 can reproduce and indicate the collecting position 12 on the culture support 2 and/or such as to automatically indicate, or enable manually defining, dimensions and/or an outline profile of a predetermined shape 13 about the collecting position 12.

The apparatus 1 can further comprise at least a first camera 17 arranged at least at the first interface 3 and connected to the first computer 9 so as to supply the first software program 14 with a second reference image of the culture support 2 housed in the first interface 3.

The first software program 14 can be configured so as to compare the first image 15 and the second image and automatically determine, or enable an operator to manually determine, a corrective factor of angular positioning of the culture support 2 in the first image 15 with respect to the second image. The first software program 14 can be configured such as to automatically correct the collecting position 12 indicated by the signaling device 11 on the culture support 2 in consideration of the corrective factor. The first camera 17, or a second camera 18, can be configured such as to record images of at least the transport support 7 during the positioning of a plurality of samples of material, therefore recording the step of depositing of the sample and/or the presence of the above-mentioned light signal 10 at the depositing sites 8 in which the material is deposited.

The second camera 18 can be connected to the first computer 9 so as to transfer the images to the first computer 9, which is aimed at conserving and/or verifying the images to confirm the real arrangement of the plurality of samples of material on the transport support 7.

The apparatus 1 can further comprise an automatic movement device, for example a motorised turntable, of the culture support 2 housed in the first interface 3 so as correct the angular position thereof so that the positioning in a third image detected by the first camera 17 corresponds to the position in the first image 15.

The apparatus 1 can further comprise at least a display 19 connected to the computer 9 and commanded by the first software program 14 for displaying at least the first image 15 and/or the second image and/or the third image of the culture support 2, singly (as illustrated in FIG. 10) or reciprocally flanked or superposed, so as to facilitate the correction of the eventual angular displacement of the culture support 2 in the first interface with respect to the position in the first image 15.

The transport support 7 can further comprise at least an electronic storage device 20, recordable and aimed at storing a plurality of data relating at least to the samples of material and the specific arrangement of each of the samples of material on the transport support 7, at each of the depositing sites 8.

The storage device 20 is constrained to the transport support 7 so as to be transportable together with the transport support 7. The plurality of stored data on the storage device 20 can comprise, for each sample or analyte, at least a datum relating to the nature or type of sample and/or the origin of the sample and at least a datum relating to the specific position of the sample on the transport support 7 or the specific depositing site in which the sample is arranged on the transport support 7.

The storage device 20 can be further provided with at least a single and individual identifying datum of the transport support 7, such as an identifying and individual serial number of the support, for example stored in an area or portion of memory dedicated to the ex-factory data and not rewritable, substantially permanent and not cancellable. In this case the transport support 7 can be without a conventional identifying label or bar code, as it can be unmistakably identified automatically by the apparatus 1 by means of the identifying datum. Alternatively, the transport support 7 can be provided with an identifying label or bar code and thus be individually identified by the apparatus 1 by means of a single and individual identifying datum detectable by the apparatus 1 by reading the bar code or by manual entering of the datum.

The storage device 20 can be further provided with one or more items of data relating to technical characteristics of the transport support 7, selected from among the following: the type of support, the type of surface of the support, the number of depositing sites 8 of the support, the arrangement of the depositing sites 8 on the support, the coordinates of the depositing sites 8 on the support, the presence or not of culture media on the support. These items of data are also for example memorized in the portion of memory dedicated to the ex-factory and not rewritable specifications.

The storage device 20 can be of the cancellable and rewritable type, for a plurality of times, so as to enable selectively cancelling and rewriting the plurality of items of data, after the completion of each series of analyses. The single identifying datum is preferably memorized permanently and is therefore not cancellable. The storage device 20 can comprise at least a TAG or a recordable RFID transponder, provided at least with an electronic memorizing chip and an RFID antenna. The storage device 20 can be a TAG or an RFID transponder of a passive type, which is powered from an external energy source during the reading or writing of data. In a variant, the storage device 20 might comprise a memory card of a non-RFID electronic type.

In a further variant, the storage device 20 might be of another type suitable for the aim. The storage device 20 can be integrated in the transport support, for example internally of the transport support 7. The transport support 7 can exhibit a flat, continuous and non-porous surface at and near to the mounting point of the storage device 20 on the transport support 7.

These characteristics are particularly important in a case in which the transport support 7 is to operate in a vacuum, for example in a case in which it is a plate for MALDI TOF analysis.

The transport support 7 can be made in a single piece. The storage device 20 can be housed internally of the transport support 7. In a variant the transport support 7 can comprise a first half-body in which the depositing seatings 8 are defined and a second half-body in which the storage device 20 is housed, the first and the second half-bodies being reciprocally constrained so as to define the transport support 7. In this case the storage device 20 is preferably housed at an internal portion of the second half-body, facing towards the first half-body, so as to be closed between the two half-bodies of the overall transport support 7. The transport support 7 can of a re-usable type for a plurality of times. In this case the transport support 7 can be realized for example of a metal material, for example steel. In a variant, the transport support 7 can be of the single-use type. In this case the transport support 7 can be realized for example of a plastic material. The transport support 7 can exhibit a matrix arrangement of depositing sites 8. The sites can be arranged in an ordered array on the transport support 7, for example in a plurality of rows and columns, in honeycomb order, in concentric circumferences or in any other ordered arrangement. The depositing sites 8 can be defined and delimited in a finite number on the transport support 7, as in the figures, and can also be numbered or indicated in another way. The sites can be constituted by wells fashioned on the transport support 7 for housing the samples of biological or microbiological material 16. The depositing sites 8 can also not be defined in a detectable way on the transport support 7, and be defined only following the depositing order of the samples on the transport support 7 or by means of a light signal, as will be described in the following. In this case the depositing sites 8 are characterised and defined only by the depositing coordinates of the samples.

The transport support 7 can be a support plate, for example a MALDI TOF mass spectrometry plate (illustrated in detail in FIG. 9). The transport support 7 can alternatively be provided with at least a culture medium for microorganisms at least at some of the depositing sites 8. The second interface 6 can comprise at least a support portion 21 able to house the transport support 7 and at least a transmission device 22 aimed at reading and modifying the contents of the electronic storage device 7 of the transport support 7.

FIG. 10 illustrates the transport support 7 mounted on the second interface 6. The transmission device 22 can comprise at least an RFID transceiver provided with at least an antenna for reading and/or writing the contents of the electronic storage device 20, of the RFID type. If the storage device 20 is passive, the second interface 6 is able to supply, in a known way, the energy required for the functioning thereof in reading and writing. The first computer 9 can be operatively connected to the second interface 6 and the first memorized software program 14 and when operating on the first computer 9 can manage the functioning of the second interface 6. The first software program 14 can command the carrying out at least of some of the process steps which will be described herein below.

The second interface 6 can comprise a support portion 21 adaptable for housing a plurality of types of different transport supports 7. The first computer 9 can comprise, for example, a control card operatively connected to the second interface 6, and a touch-screen panel operatively connected to the control car and aimed at commanding functioning thereof. The first software program 14, or a portion of additional software, can be installed and operate on the touch-screen panel. The computer can further comprise, in addition or alternatively to the touch-screen panel, a common PC or another type of computer or data system interfaced to the control card and on which the software program operates. In a variant a further storage device 20 can be included, of the above-described type, associated to the culture support 2, and in this case the first interface 3 can exhibit like characteristics to the second interface 6 as far as the reading and/or writing system on the storage device 20 is concerned.

The invention further relates to a process for treating samples of biological or microbiological material 16, comprising at least a step of arranging a culture support 2, in particular a Petri dish, in a first housing interface 3 of an apparatus 1 for treatment of samples of biological or microbiological material 16, at least a biological or microbiological material 16 being present on the culture support 2.

The process can also comprise a step of projecting on the culture support 2 housed in the first interface 3 at least a light signal 10 so as to visually indicate at least a collecting position 12 of the biological or microbiological material 16 on the culture support 2.

The process can further comprise a step of collecting a sample of biological or microbiological material from the culture support 2 at a collecting position 12.

The process can further comprise a step of obtaining at least a first image 15 of the culture support 2 having the biological or microbiological material 16, before the step of collecting the sample.

The process can further comprise the step of selecting, in the first image 15, the collecting position 12 of a sample of biological or microbiological material 16.

The process can further comprise the step of automatically or manually defining dimensions and/or a predetermined shape 13 to be projected by means of the light signal about the collecting position 12 arranged in the first interface 3.

The process can further comprise a step of obtaining at least a second reference image of the culture support 2 housed in the first interface 3 and comparing the first image 15 with the second image.

The process can further comprise a step of automatically or manually determining a corrective factor of angular positioning of the culture support 2 in the first image 15 with respect to the second image.

The process can further comprise the step of automatically correcting the collecting position 12 indicated by the signaling device 11 on the culture support 2 in consideration of the corrective factor.

The process can further comprise a step of automatically or manually correcting the angular position of the culture support 2 in the first interface 3 in such a way as to annul the corrective factor and make the angular position of the culture support 2 coincide in a third image detected by the first camera 17 with the corresponding angular position of the culture support 2 in the first image 15.

The process can further comprise a step of arranging a plurality of samples of biological or microbiological material 16 on a corresponding plurality of depositing sites 8 of a transportable transport support 7. The depositing of the samples can be done in a known way, for example by seeding microbiological samples, collected from the culture support 2 or by means of depositing biological samples diluted in a liquid.

In substance, the process, for the part regarding the management of the culture support 2 and the collection of samples to be analysed thereby, can be done for example in the following way.

The culture support 2, provided with a suitable culture medium, is first seeded in a known way, manually or automatically, by means of depositing of a biological material 16 comprising microbiological material, typically bacteria. The culture support 2 is then subjected to a predetermined incubation period, in order to obtain the growth and isolation of bacterial colonies thereon. At this point at least a high-definition first image 15 is obtained of the specific culture support 2 preliminarily-identified, for example by reading a bar code, and arranged with a precise angular orientation, by means of a camera, and a doctor or highly-specialised operator can identify and select at least a collecting point, corresponding to at least a bacterial colony developed on the culture support 2 by means of a first computer 9 and a first software program 14, or by means of a preliminary computer and a preliminary software program.

In the second case the first image 15, with at least the collecting point selected, is transferred to the apparatus 1 for the treatment of biological and/or microbiological material 16 according to the present invention, so as to be processed by the first computer 9 and by the first software program 14. The culture support 2 is then physically transported to the laboratory in which the collecting of a sample takes place and the transfer thereof onto a transport support 7 is performed. The culture support 2 is then newly identified individually, for example by means of a bar code reader, and is housed in the first interface 3 by aligning the bar code or other reference of the culture support 2 with a reference indicator 4 of the first interface 3, so as to define a known angular position of the culture support 2. At this point a second culture support 2 can be obtained by means of the first camera 17, and a comparison can be made of the first image 15 with the second image so as to verify the correctness of the angular position of the culture support 2 in the first interface 3. At this point automatic or manual correction can be made of the position of the culture support 2, or correction using the software program of the collecting position 12 in consideration of the angular displacement between the two images.

The apparatus 1 can then indicate, by means of the laser beam 10, the corrected collecting position 12 on the culture support 2, so as to enable an operator to exactly collect the desired sample by the doctor, and then position the sample on a transport support 7 or in any case send the sample towards the desired analysis. In this way the risk of error in collecting the sample selected by the doctor prior to the collecting moment can be eliminated or significantly reduced.

The process for treating the samples of biological or microbiological material 16 can further comprise the step of memorizing, on an electronic storage device 20 constrained to the transport support 7 so as to be transportable together with the transport support 7, a plurality of data relating at least to the samples of biological or microbiological material 16 and the specific arrangement of each thereof on the transport support 7, at the depositing sites 8.

The process can further comprise a step of reading, from the electronic storage device 20, constrained to the transport support 7 so as to be transportable together with the transport support 7, at least the plurality of data relating at least to the samples of biological or microbiological material 16 and to the specific arrangement to be carried out for each thereof on the transport support 7, at the depositing sites 8.

The process can further comprise a step of reading from the electronic storage device 20, constrained to the transport support 7 so as to be transportable together with the transport support 7, at least a single and individual identifying datum of the transport support 7, in such a way as to surely identify, and automatically, each specific transport support 7. It is possible that during the step of reading the electronic storage device 20, only the mentioned single and individual identifying datum of the transport support 7 is read; for example in a case in which the memorization of the plurality of data relative to the samples and the arrangement thereof is done during the depositing of the samples.

The step of reading from the electronic storage device 20 is preferably carried out before the step of depositing a plurality of samples of material on the transport support 7, both possibly for reading the mentioned identifying datum, and possibly for reading the data relative to the arrangement to be carried out.

The process might not include any step of reading from the electronic storage device 20 the plurality of data and/or the identifying datum, during the depositing of the sample in a case in which the transport support 7 is identified by means of another means and the working scheme is established together with the depositing of the samples.

The step of memorising on the electronic storage device 20 can be carried out before the step of arranging a plurality of samples of material and before the step of reading from the electronic storage device 20. The step of memorizing on the electronic storage device 20 can further be carried out in a different place from the place in which the step of arranging a plurality of samples of material and/or the step of reading by the electronic storage device 20 occurs. This happens for example in a case in which a first data system is used to plan the arranging of the samples on the transport support 7, while recording the work plan on the storage device 20 of the transport support, and then the transport support 7 is transferred to a second data system, at which the storage device 20 is read so as to enable identification of the transport support 7 (which might occur also using conventional bar codes or another known method) and the reading of the work plan with the arrangement of the samples which is to be carried out on the transport support 7. Alternatively, the step of memorizing on the electronic storage device 20 can be carried out at the same time as the step of arranging the plurality of samples of material on the transport support 7, for example in a case in which the work plan with the arrangement of the samples on the transport support 7 is not imported from another system, but is established directly before or during the depositing of the samples, recording on the storage device 20 so as to enable a successive reading, for example in combining sites with the results of the analysis or recording thereof on the storage device 20.

In a variant, the step of memorising on the electronic storage device 20 can further be carried out following the step of arranging a plurality of samples of material on the transport support 7. Briefly, the work plan can initially be designed in a laboratory processing system, and then the work plan can be realized using a management software program and the work plan can be stored on the storage device 20 of the transport support 7. At this point the step of guided depositing of the samples can be carried out according to the established work plan. Then, in another place, the recognition of the prepared transport support 7 can take place and the contents thereof and the transport support 7 can be subjected to the further analyses of the case, for example a MALDI-TOF analysis in a relative apparatus for spectrometry analysis, with automatic combination by the data system of the results to the respective samples.

The process can further comprise a step of cancelling the plurality of data from the electronic storage device 20 and/or updating and/or replacing them with a corresponding plurality of updated or modified information, for example in order to enable re-use of the transport support 7. This data can be, before cancellation from the storage device 20, recorded on the computer or on another data system so as to enable entry in a historical database thereof and re-use for carrying out further research or statistics.

The process can further comprise a step of projecting on the transport support 7 at least a light signal, during the step of arranging a plurality of samples of material at the effective depositing position or sites 8 in which each sample of material is deposited, so as to guide the positioning or so as to visibly indicate the positioning.

The process can further comprise a step of recording images or photographs at least of a part of the transport support 7 during the step of arranging a plurality of material samples, and conserving and/or verifying the images to confirm the real arrangement of the plurality of samples of material on the transport support 7. In substance the process, as it regards the management of the transport support 7 and the depositing of the samples thereon, can be realized in various ways, among which for example the following: 1) it is possible to prepare on a first data system a work plan for the depositing of the samples, record the work plan on the storage device 20 of the transport device 7, by means of a preliminary interface, and then transfer the transport support 7 into the laboratory in which the samples are to be deposited. In the laboratory the transport support 7 is coupled with a second interface 6 which enables identification of the transport support 7 and the reading of the work plan, which is then carried out by depositing, possibly automatically, the samples on the device. The depositing can be guided by a light signal which indicates the sites where the sample is to be deposited, and photographs can be taken of the images to confirm the correct depositing of each sample. The transport support 7 thus-prepared can then be transferred into the place where the further analysis of the samples is to be carried out, with the possibility of newly reading the work plan memorized on the device and automatically combining the results of the analysis to the plurality of data relating to the samples, possibly also memorizing them on the storage device 20, or on the local data system; 2) the work plan can be established directly by the first software program 14 or another additional software program present in the laboratory in which the samples are deposited, and in this case first the transport support 7 is identified (by means of the bar code or the single datum of the storage device 20), after which the samples are deposited and at the same time or following this, the data relating to the samples and the location thereof are memorized on the storage device 20. Thereafter the same operations as for the first option are carried out. 3). In a variant, it is also possible to carry out a substantially free depositing of the samples on the transport support 7, detecting and recording on the storage device 20 the information relating to the samples and the depositing thereof while it is carried out, or immediately after the depositing.

The present invention enables obtaining one or more of the following advantages.

Firstly, the invention enables obviating the problems encountered in the prior art.

The invention also enables eliminating or at least significantly reducing the risk of human errors in the carrying out of the analysis procedures and/or in the determining of the results thereof.

The invention further provides a high degree of reliability and repeatability of the results, and provides an apparatus and a process for the treatment of samples or analytes which are extremely flexible and adaptable to various operative requirements and to various types of analysis.

The invention further enables a simplification of the treatment processes of the data relative to the analyses of the samples analysed, and further enables a portability of the data among different data systems that are not connected to one another.

The invention further comprises a complete traceability of the historical information relating to the analyses carried out and the devices and subjects involved, and enables obtaining research and statistics on the results which enable further increasing the quality of the analyses carried out and the safety of the environments.

The invention further enables significantly increasing the safety of the analysis processes of the samples, the security of the patients and the security of the various subjects involved in the analyses.

Lastly, the invention is simple and economical to actuate.

1. An apparatus for treating samples of biological or microbiological material (16), wherein the apparatus (1) comprises:
a first housing interface (3) for a culture support (2), a biological or microbiological material (16) being arranged on the culture support (2);
a first computer (9) provided with a processor and a memory unit;
a visual signaling device (11), operatively connected to the first computer (9);
a first software program (14) stored and operating on the first computer (9) and configured such as to process a memorized first image (15) of the culture support (2), wherein the first image (15) exhibits a selected position (12) for collecting a biological or microbiological material (16), the first software program (14) being configured to control functioning of the visual signaling device (11) so as to emit a light signal on the culture support (2) housed in the first interface (3) and visually indicate the collecting position (12) of a biological or microbiological material (16) on the culture support (2), so as to guide a collecting of a sample of the biological or microbiological material (16) by an operator at the collecting position (12).

2. The apparatus of claim 1, wherein the apparatus is configured such as to receive the first image (15) with the collecting position (12) already previously selected by a user by a second software program operating on a second computer.

3. The apparatus of claim 1, wherein the first software program (14) is configured so as to display and enable a user to select the collecting position (12) on the first memorized image (15) of the culture support (2).

4. The apparatus of claim 2, wherein the first software program is configured so as to automatically define or to enable manually defining dimensions and/or a profile of a predetermined shape (13) about the collecting position (12).

5. The apparatus of claim 1, wherein the visual signaling device (11) is aimed at emitting a laser beam (10) pointed towards a position that is proximal to and distinct from the collecting position (12).

6. The apparatus of claim 1, wherein the visual signaling device (11) is aimed at generating a laser beam (10) defining a predetermined shape (13) developing about the collecting position (12).

7. The apparatus of claim 6, wherein the predetermined shape (13) is defined statically by the laser beam (10).

8. The apparatus of claim 6, wherein the predetermined shape (13) is defined dynamically by the laser beam (10).

9. The apparatus of claim 1, further comprising a second housing interface (6) for a transport support (2) of biological or microbiological samples (16), the transport support being provided with a plurality of depositing positions or sites (8) able each to receive a sample of biological or microbiological material (16).

10. The apparatus of claim 1, wherein the visual signaling device (11) is commanded by the first software program (14) of the first computer (9) such as further to indicate a depositing position or site on the transport support (7) or to progressively indicate the depositing positions or sites (8) of the samples on the transport support (7).

11. The apparatus of claim 1, wherein the visual signaling device (11) is configured to emit a laser beam (10) selectively indicating a depositing position or site or a plurality of depositing positions or sites (8) on the transport support (7).

12. The apparatus of claim 1, wherein the first housing interface (3) is provided with a reference indicator (4) aimed at enabling a precise positioning according to a defined orientation of the culture support (2) on the first housing interface (3).

13. The apparatus of claim 12, wherein the reference indicator (4) is determined such as to enable arranging a reference element (5) of the culture support (2), at the reference element (5) with the culture support (2) arranged in the first housing interface (3).

14. The apparatus of claim 1, further comprising a first camera (17) arranged at the first interface (3) and connected to the first computer (9) so as to supply to the first software program (14) a second reference image of the culture support (2) housed in the first interface (3), the first software program (14) being configured to compare the first image (15) and the second image and to automatically determine, or to enable an operator to manually determine, a corrective factor of angular positioning of the culture support (2) in the first image (15) with respect to the second image.

15. The apparatus of claim 14, wherein the first software program (14) is configured such as to automatically correct the collecting position (12) indicated by the signaling device (11) on the culture support (2) in consideration of the corrective factor.

16. The apparatus of claim 15, wherein the apparatus (1) further comprises an automatic movement device of the culture support (2) housed in the first interface (3) such as to correct the angular position thereof so that the positioning in a third image detected by the first camera (17) corresponds to the positioning thereof in the first image (15).

17. The apparatus of claim 14, further comprising a display (19) connected to the computer (9) and commanded by the first software program (14) for displaying the first image (15) and/or the second image and/or the third image of the culture support (2).

18. A process for treating samples of biological or microbiological material (16), comprising steps of:
predisposing a culture support (2) exhibiting biological or microbiological material (16) that has grown thereon;
obtaining a first image (15) of the culture support (2);
selecting, in the first image (15), a collecting position (12) of a sample of biological or microbiological material (16);
arranging the culture support (2) in a first housing interface (3) of an apparatus (1) for treatment of biological or microbiological samples (16);
projecting, onto the culture support (2) housed in the first interface (3), a light signal (10) such as to visually indicate the collecting position (12) of the biological or microbiological material (16) on the culture support (2); and
collecting the sample of the biological or microbiological material (16) from the culture support (2) at the collecting position (12).

19. The process of claim 18, wherein the steps of obtaining the first image (15) and selecting the collecting position (12) in the first image (15) are carried out by a preliminary computer and a preliminary software program by an operator.

20. The process of claim 19, wherein the first image (15), with the collecting position (12) selected, is transferred to the apparatus (1) before projecting the light signal (10) onto the culture support (2).

21. The process of claim 18, comprising:
automatically, or manually, defining dimensions and/or a profile of a predetermined shape (13) to be projected by the light signal about the collecting position (12) arranged in the first interface (3).

22. The process of claim 18, comprising:
obtaining a second reference image of the culture support (2) housed in the first interface (3) and comparing the first image (15) with the second image;
automatically, or manually, determining a corrective factor of angular positioning of the culture support (2) in the first image (15) with respect to the second image;
automatically correcting the collecting position (12) indicated by the signaling device (11) on the culture support (2) in consideration of the corrective factor;
automatically or manually correcting the angular position of the culture support (2) in the first interface (3) such as to annul the corrective factor and make the angular position of the culture support (2) coincide in a third image detected by a first camera (17) with the corresponding angular position of the culture support (2) in the first image (15).
